(19) United States
(12) Patent Application Publication
(10) Pub. No.: US 2022/0370784 A1
(43) Pub. Date: Nov. 24, 2022

(54) MEDICAL SYSTEM COMPRISING AN IMPLANTED INTERNAL UNIT, AN EXTERNAL UNIT, AND A METHOD OF INITIATING OPERATION OF EXTERNAL UNIT

(71) Applicant: InvivoPower AB, MÖLNDAL (SE)

(72) Inventor: Bengt Bern, MÖLNDAL (SE)

(21) Appl. No.: 17/765,931

(22) PCT Filed: Oct. 27, 2020
(86) PCT No.: PCT/SE2020/051040

§ 371 Date: Apr. 1, 2022

(30) Foreign Application Priority Data
Nov. 7, 2019 (SE) 1951276-3

(51) Int. Cl. A61M 60/122; A61M 60/178

(52) U.S. Cl. CPC A61M 60/122; A61M 60/178; A61B 2560/0219; A61F 2250/0001

(57) ABSTRACT

A method of initiating operation of an external unit for a medical system further comprising an internal unit implanted into a body of a patient; a transformer core arranged under the skin of the patient; and internal cabling connecting the internal unit and the transformer core, the internal cabling comprising an internal winding around the transformer core, wherein the external unit comprises external cabling including an external winding around the transformer core to allow supply of power from the external unit to the internal unit via the transformer core, the method comprising the steps of: evaluating, by the external unit, a signal indicative of a magnetic flux in the transformer core; when the signal indicates that the magnetic flux in the transformer core is below a predefined threshold flux, providing power to the internal unit by the external unit via the transformer core.

### FIELD OF THE INVENTION

The present invention relates to a medical system, to an external unit comprised in the medical system, and to a method of initiating operation of the external unit.

### BACKGROUND OF THE INVENTION

Medical devices having one or more implantable units, generally referred to as implantable medical devices, have provided a wide range of benefits to patients over recent decades. In particular, devices such as implantable hearing aids, implantable pacemakers, defibrillators, eye implants, retina implants, heart pumps, ventricular assist devices, total artificial hearts, drug delivery systems, gastric implant, nerve stimulators, brain stimulators, functional electrical stimulation devices, such as cochlear prostheses, organ assist or replacement devices, and other partially or completely-implanted medical devices, have been successful in performing life-saving and/or lifestyle enhancement functions for a number of years.

As such, the type of implantable devices and the range of functions performed thereby have increased over the years. For example, many such implantable medical devices often include one or more instruments, apparatus, sensors, processors, controllers or other functional mechanical, electrical or electronic components that are permanently or temporarily implanted in a patient to perform diagnosis, prevention, monitoring, treatment or management of a disease or injury or symptom thereof, or to investigate, replace or modify of the anatomy or of a physiological process. Many of these implantable components receive power and/or receive data and/or transmit data over a wireless transcutaneous link from and/or to external units that are part of, or operate in conjunction with, the implantable unit.

The wireless transcutaneous link is conventionally realized as an inductive link, with an external unit comprising a transmitter winding and an implantable unit comprising a receiver winding. Typically, the receiver winding is implanted below the skin, and the transmitter winding is attached to the patient skin opposite to the implanted receiver winding such that the two windings are in parallel planes on both sides (external and implantable positions) of the skin. These systems are typically referred as TET links (TET-Transcutaneous Energy Transfer). For TET links it is rather difficult to fixate and position the transmitter winding to the skin of a patient. Gluing solutions and special vests to fixate/position the transmitter winding have been tried but have had a lot of problems especially when the patient has been sleeping. For life sustaining applications like heart pumps, ventricular assist devices or total artificial hearts this fixation/positioning fixation and positioning is very critical. If the transmitter winding falls off or if it is in the wrong position the transcutaneous power transfer is affected and could in worst case be life threatening for the patient. Therefor TET systems for life sustaining applications are almost always implemented with an implantable battery. If the transmitter winding falls off or if it is in the wrong position the implantable battery will continue to supply power to the implantable unit. The implantable unit could be as an example a heart pump. However implantable batteries give rise to a new problem. Today's battery technology has a limited number of recharging cycles. For an example if the battery recharging is limited to one thousand charging cycles and the patient charges the implantable battery two times a day the battery needs to be replaced every one and a half years. Frequent replacement of the implantable battery requires costly surgery, increases the risk for infection for the patient and reduces the quality of life for the patient due to repeated hospital stays. Therefor there is a large need for a transcutaneous energy supply system where the transmitter winding doesn't fall off or gets into the wrong position and making it possible to keep the battery that powers the system on the outside of the patient. Further there is a need, in such a system, to be able to change the external unit, containing the battery on the outside of the patient, seamlessly and safely without any loss in the energy supply to the internal unit/heart pump. In the exchange of the external unit the initiation process of the external unit will be very important to have a seamless and safe exchange. The need to change the external unit, containing the battery, seamlessly and safely is not only for medical systems comprising heart pumps like ventricular assist devices or total artificial hearts, the need is for all types of life sustaining medical systems.

In view of the above, it would thus be desirable to provide for an improved external unit and method of initiating operation of such an external unit.

### SUMMARY

It is an object of the present invention to provide for an improved external unit and method of initiating operation of such an external unit.

According to a first aspect of the present invention, it is therefore provided a method of initiating operation of an external unit for a medical system further comprising an internal unit implanted into a body of a patient; a transformer core arranged under the skin of the patient; and internal cabling connecting the internal unit and the transformer core, the internal cabling comprising an internal winding around the transformer core, wherein the external unit comprises external cabling including an external winding around the transformer core to allow supply of power from the external unit to the internal unit via the transformer core, the method comprising the steps of: evaluating, by the external unit, a signal indicative of a magnetic flux in the transformer core; when the signal indicates that the magnetic flux in the transformer core is below a predefined threshold flux, providing power to the internal unit by the external unit via the transformer core.

According to a second aspect of the invention, it is provided an external unit for a medical system further comprising an internal unit implanted into a body of a patient; a transformer core arranged under the skin of the patient; and internal cabling connecting the internal unit and the transformer core, the internal cabling comprising an internal winding around the transformer core, wherein the external unit comprises: external cabling configured to allow formation of an external winding around the transformer core; sensing circuitry coupled to the external cabling and configured to sense a magnetic flux in the transformer core when the external cabling forms the external winding around the transformer core; and power supply circuitry coupled to the external cabling and configured to supply power to the internal unit via the transformer core when the sensing circuitry is sensing the magnetic flux in the transformer core, and the magnetic flux is below a predefined threshold flux.

The present invention is based on the realization that convenience and safety can be improved by configuring the external unit of the medical system in such a way that the initiation process for the external unit of the medical system is the same whether it is a first time initiating the external unit or if it is a seamless exchange from one external unit to another external unit. If it is the first time initiating the external unit, the external unit is connected to the patient by feeding the external cabling through the pierced skin tunnel of the patients. The connector is closed forming the external winding of the medical system. Then the external unit is activated and the external unit measures if there is any time varying magnetic flux in the implanted transformer core. Since there is no other external unit connected to the transformer core of the patient there is no time varying magnetic flux in the transformer core. Because the magnetic flux in the transformer core is below a predefined threshold flux the external units starts to provide power to the internal unit via the transformer core.

If one wants to do a seamless exchange from one external unit to another external unit it will be the following sequence: A first external unit is already connected to the patient providing power to the internal unit via the transformer core. A secondary external unit is connected to the patient by feeding the external cabling through the pierced skin tunnel of the patient. The connector is closed forming a second external winding. Then the secondary external unit is activated and the secondary external unit measures if there is any time varying magnetic flux in the implanted transformer core. Since the first external unit is providing power to the internal unit via the transformer core there is time varying magnetic flux in the transformer core. The second external unit then just continues to evaluate/measure the magnetic flux in the transformer core. Then the first external unit is removed from the transformer core. The second external unit then concludes that the signal that indicates that the magnetic flux in the transformer core is below a predefined magnetic flux threshold and then second external unit immediately starts to provide power to the internal unit via the transformer core. The two external units have thus been exchanged seamlessly without any loss of power in the internal unit. Because the initiation process is the same for both first time initiating the external unit and exchanging the external unit, it reduces the risk greatly that an operator makes an operational error during the start-up/exchange initiation of the external unit. This is extremely important for life sustaining medical systems.

In one embodiment the internal unit comprises one or more of processing unit, rechargeable battery, vibrator, vibratory unit, electrode array, pump, sensor, drug, heart pump, ventricular asst device, total artificial heart.

The internal unit thus may be selected from a group consisting of one or more of:

i) an implantable hearing aid comprising a cochlear implant comprising an implantable electrode array configured to be positioned within a cochlea of the user, the electrode array being configured to deliver electrical charges in accordance with the output,

ii) an implantable hearing aid comprising an auditory trans modal implant comprising an implantable electrode array configured to be positioned within a modiolus of the user, the electrode array being configured to deliver electrical charges in accordance with the output,

iii) an implantable hearing aid comprising an auditory brainstem implant comprising an implantable electrode array (typically provided as a pad) configured to be implanted directly onto brainstem, the electrode array being configured to deliver the electrical charges in accordance with the output,

iv) an implantable hearing aid comprising a bone conduction hearing aid comprising an implantable vibrator configured to be attached to skull of the user, the vibrator being configured to generate vibrations in accordance with the output,

v) an implantable hearing aid comprising a middle ear implant comprising a vibratory unit configured to attach to one of the bones of the middle ear and/or to one of the windows of the cochlea, the vibratory unit being configured to generate vibrations in accordance with the output,

vi) an artificial pacemaker comprising an electrode array configured to deliver electrical charges in accordance with the output.

vii) an implantable heart pump such as an impeller, a ventricular assist device (VAD) or a total artificial heart (TAH) comprising a pump configured to be attached to a user's heart, the pump being configured to provide blood flow within user's body, and

vii) an implantable drug delivery system comprising an implantable capsule comprising a drug and a pump that is configured to attach to the implantable capsule and release, through a pumping action, a predefined amount of drug from the capsule to the user's body. In this embodiment, the drug delivery system may further include an implantable sensor that is configured to capture a biological data such as blood glucose level. The implantable processing unit is configured to receive the biological data and compare the received data with a stored normal range to determine a difference and accordingly, based on the difference, determine the amount of drug (predefined amount) to be released. The normal range, along with difference to amount of releasable drug may be stored as a look up table in a memory that the processing unit is configured to access. The processing unit is further configured, based on the determined predefined amount, to activate the pump (adapted to operationally connect to the capsule) for a duration that lets the predefined amount of the drug to be released from the capsule,

viii) implantable deep brain stimulators or implantable nerve stimulators comprising an implantable electrode array configured to be implanted directly or indirectly onto the brain or nerve respectively, the electrode array being configured to deliver the electrical charges in accordance with the output comprising a stimulation pulse. The delivered electrical charges may be utilized to provide brain with information,

viii) eye implants or retina implants comprising a camera for capturing images and an implantable electrode array configured to deliver electrical charges in accordance with the captured images,

ix) an implantable cardioverter defibrillator comprising an electrode array (usually as electrical pads) configured to deliver electrical charges (for example by way of electrical shock) in accordance with a comparison of monitored rate and rhythm of the heart with a preset number. In this embodiment, the defibrillator may also include sensors in order to monitor the rate and rhythm of the heart,

x) an implantable gastric stimulator configured to be implanted in an abdomen of the user and comprising an electrode array that is configured to deliver electrical charges (typically by way of mild electrical pulses) to nerves and smooth muscle of lower stomach of the user, and

xi) an implantable brain computer interface system comprising an implantable sensor adapted to capture neural signals in response to brain activity.

xii) an implantable battery.

One or more of the above paragraphs refers to use of a medical device or a combination thereof. For example, a cochlear implant may be used alone but may also be used in combination such as providing mechanical stimulation by way of bone conduction hearing aid at a first ear and an electrical stimulation by way of cochlear implant at a second ear of the user. In another example, the same user may be utilizing a cochlear implant as well as an implantable cardioverter defibrillator. Other such examples of combinations are within the scope of embodiments of the invention.

In an embodiment the transformer core is preferably a solid continuous loop that is made up of a magnetic, like ferrite, material preferably having a high magnetic permeability. Such high magnetic permeability is at least 10, preferably at least 100, more preferably at least 1000. Such material may be selected from a group consisting of a ferrite material, and soft iron. Other commercially available products under brand names VACOFLUX™, VACODURT™, VOCADUR S PLUS™, TRAFOPERM™, CRYOPERM™, PERMAX™, PERMENORM™ ULTRAPERM™, VACOPERM™, CHRONOPERM™, MEGAPERM™, MUMETALL™, RECOVAC™, and THERMOFLUXT™, as produced by Vacuumschmelze GmbH & Co. KG or REMKO™ as produced by Uddeholm A/S may also be used. As the transformer core is made up of a material having a high magnetic permeability, most of the flux lines are concentrated within the magnetic material and thus allow for a high coupling coefficient between the external winding and the internal winding. It would be apparent to the skilled person that a material of different magnetic permeability may also be used so long as the material has high enough magnetic permeability to guide the flux lines generated in response to excitation of the external winding in a focused way towards the internal winding in a way explained in embodiments of the invention.

In an embodiment, the transformer core solid continuous loop is defined by a geometrical shape that includes a closed curve, defining a closed loop structure, wherein a point moving along the closed curve forms a path from a starting point to a final point that coincides with the starting point when the closed curve is in a closed mode.

In different embodiments, the transformer core solid continuous loop may include shape that is selected from a circular, elliptical, rectangular, square, polygonal shape, curved shape or a combination thereof.

In different embodiments the transformer core is a semi continuous loop with one or more narrow airgap(s).

In an embodiment the internal winding is formed by a conductive wire being wound around and along at least a part of the length of the transformer core. The number of turns of the winding could be 1-200 more preferably 1-50, and most preferably 1-20.

In an embodiment, the external cabling is configured to form the external winding through a healed pierced hole of the body such that the transformer core and the external winding are arranged in an interlocked first hopf link configuration, and the transformer core and the internal winding are arranged in an interlocked second hopf link configuration. Because of the first hopf link configuration and second hopf link configuration, a substantial number of magnetic flux lines are concentrated within the transformer core and passes through the internal winding, thereby substantially improving the coupling coefficient and the energy efficiency of the system. Further none of the external winding, the transformer core or the internal winding penetrates the human body outer skin surface.

In an embodiment, the time varying magnetic flux frequency created in the transformer core by the power supply circuitry of the external unit is preferably 5 kHz-1 MHz, more preferably 10 kHz-500 kHz and most preferably 30 kHz-300 kHz.

In an embodiment of the medical system the external unit further comprises sensing circuitry coupled to the external cabling and configured to sense a time varying magnetic flux in the transformer core when the external cabling forms the external winding around the transformer core. By time multiplexing the connection of the external winding it is possible to measure the induced voltage in the external winding and to calculate the time varying magnetic flux in the transformer core according to principle of Faraday's law. Further it is possible, using process circuitry in the external unit, to control the power supply circuitry to supply power to the internal unit via the transformer core when the magnetic flux in the transformer core is lower than a predetermined value.

In an embodiment of the medical system the external unit further comprises an indicator when power is being provided by the external unit to the internal unit. The indicator could be visual for example an LED or a sound or a tactile indication.

In summary, the present invention relates to an external unit of a medical system and a method of initiating the external unit. The invention also presents a seamless exchange of the external unit without loss of power in the internal unit. Because the initiation process is the same for both first time starting up the external unit and the exchange of the external unit, it reduces the risk greatly that an operator makes an operational error during the start-up/exchange of the external unit. The invention presents the initiating operation method of an external unit and an external unit for a medical system further comprising an internal unit implanted into a body of a patient, a transformer core arranged under the skin of the patient and internal cabling connecting the internal unit and the transformer core, the internal cabling comprising an internal winding around the transformer core, wherein the external unit comprises external cabling including an external winding around the transformer core to allow supply of power from the external unit to the internal unit via the transformer core the method comprising the steps of:
i) evaluating, by the external unit, a signal indicative of a magnetic flux in the transformer core;
ii) when the signal indicates that the magnetic flux in the transformer core is below a predefined threshold flux, providing power to the internal unit by the external unit via the transformer core.

### BRIEF DESCRIPTION OF THE DRAWINGS

These and other aspects of the present invention will now be described in more detail, with reference to the appended drawings showing an example embodiment of the invention, wherein:

FIG. 1 illustrates the external unit of a medical system with sensing circuitry configured to sense a magnetic flux in the transformer core, according to an embodiment of the invention;

FIG. 2 illustrates the flowchart to activate the sensing circuitry according to an embodiment of the invention;

FIG. 3 illustrates a medical system according to an embodiment of the invention;

FIG. 4A illustrates a first external unit of a medical system forming an external winding connected to the body of a patient and a second external unit not connected to the body according to an embodiment of the invention;

FIG. 4B illustrates a first external unit of a medical system and a second external unit connected to the body of a patient according to an embodiment of the invention;

FIG. 4C illustrates a first external unit of a medical system forming an external winding just disconnected from the body of a patient and a second external unit connected to the body of a patient according to an embodiment of the invention; and

FIG. 5 illustrates an external unit of a medical system providing a predefined indication when power is being provided by the external unit to the internal unit according to an embodiment of the invention.

### DETAILED DESCRIPTION OF EXAMPLE EMBODIMENTS

In the present detailed description, various embodiments of the method, external unit and medical system according to the present invention are mainly described with reference to a heart pump. It should be noted that this by no means limits the scope of the present invention as defined by the claims, which also encompass, for instance, implantable hearing aids, implantable pacemakers, defibrillators, eye implants, retina implants, heart pumps, ventricular assist devices, total artificial hearts, drug delivery systems, gastric implant, nerve stimulators, brain stimulators, functional electrical stimulation devices, such as cochlear prostheses, organ assist or replacement devices, and other partially or completely-implanted medical devices.

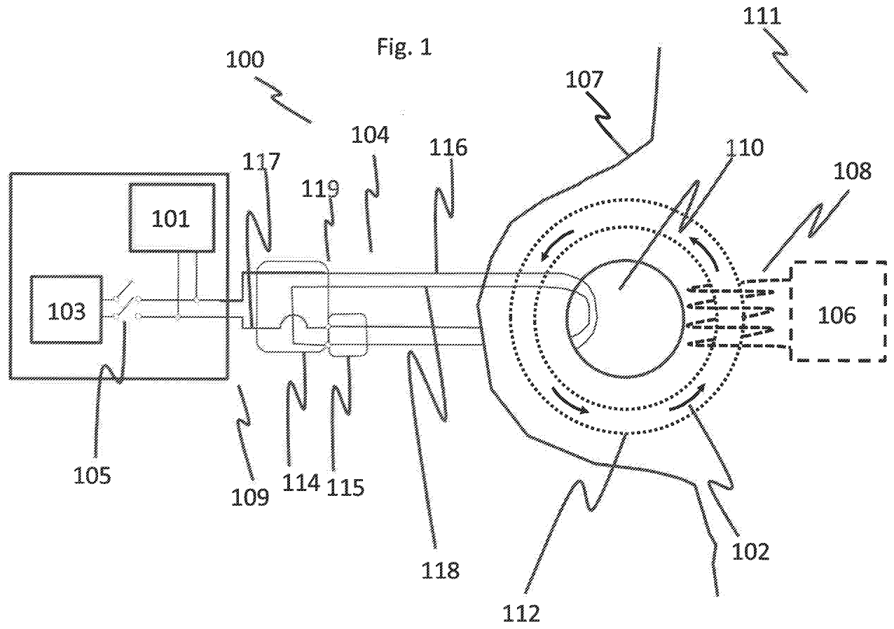

FIG. 1 illustrates the medical system 100 according to an embodiment of the invention. The medical system comprises an internal unit 106 implanted into a body of a patient; a transformer core 112 arranged under the skin 107 of the patient; and internal cabling 108 connecting the internal unit and the transformer core 112, the internal cabling comprising an internal winding around the transformer core, wherein the external unit comprises external cabling 104 including an external winding around the transformer core to allow supply of power from the external unit 109 to the internal unit 106 via the transformer core 112. Further the external cabling comprises: a connector 119 including a first connector part 114 and a second connector part 115, a first conductive current path 117 between the power supply circuitry 103 and the first connector part 114, conductively connecting the first connector part 114 and the power supply circuitry 103, a second conductive current path 116 between the power supply circuitry 103 and the second connector part 115, conductively connecting the second connector part 115 and the power supply circuitry 103 and a third conductive current path 118 between the first connector part 114 and the second connector part 115, conductively connecting the first connector part 114 and the second connector part 115, wherein the first connector part 114 and the second connector part 115 are joinable to conductively connect the first conductive current path 117 to the second conductive current path 116 via the third conductive current path 118 so that a second winding can be formed around the transformer core 112 by joining the first connector part 114 and the second connector part 115. Further the external cabling is formed through a pierced healed skin tunnel 110 of a patient 111. By using time multiplexing control, the power circuitry 103 is disconnected from the external cabling 104 using connection means 105. Further one measures and evaluates, using the sensing circuitry 101 connected to the external cabling 104, the induced voltage in the external cabling winding caused by the time varying magnetic flux 102 in the transformer core 112, using the principle of Faraday's law i.e. the time varying magnetic flux in the transformer core will generate an induced voltage in the sensing circuitry 101. By evaluating the measured induced voltage, which is a signal indicative of the time varying magnetic flux in the transformer core, one can decide to, when signal indicates that the time varying magnetic flux in the transformer core is below a predefined threshold flux, provide power to the internal unit 106 by the external unit 109 via the transformer core 112. The power is provided by closing the connection means 105 and providing a time varying current in the external cabling 104. In FIG. 1 the external winding contains two turns.

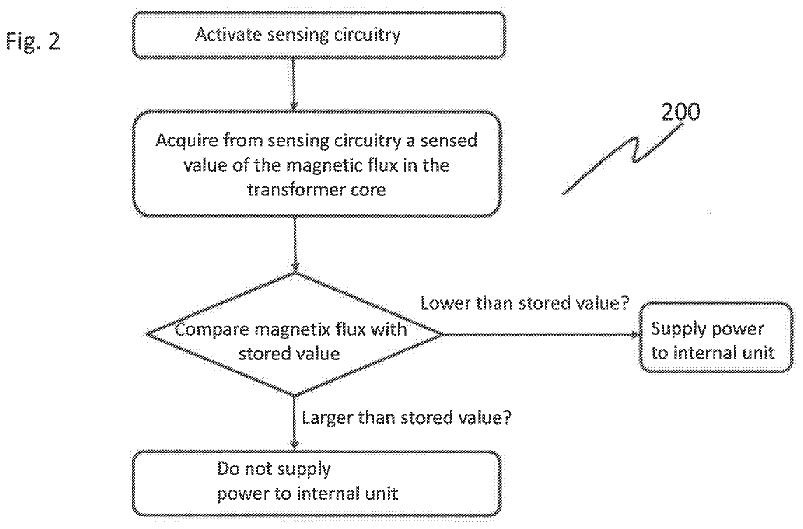

FIG. 2 illustrates the flowchart 200 of the processing circuitry configured to activate the sensing circuitry according to an embodiment of the invention. The sensing circuitry is activated to sense the time varying magnetic flux in the transformer core. The sensing circuitry returns a sensed value related to the amount of time varying magnetic flux in the transformer core. The sensed value is then compared with a stored value indicative of the threshold magnetic flux. If the sensed value is lower than the stored value the power supply circuitry is activated to supply power to the internal unit via the transformer core.

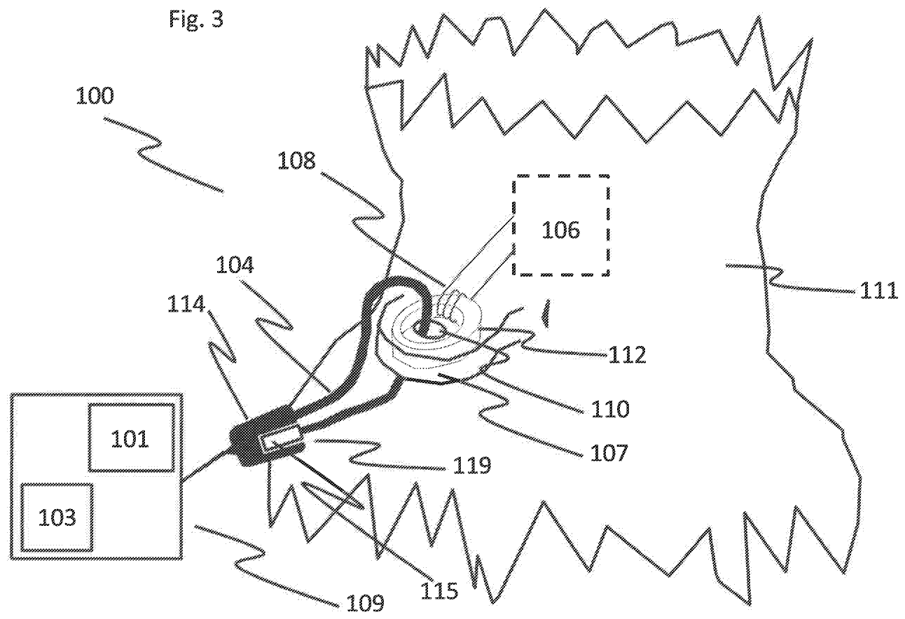

FIG. 3 illustrates the same medical system 100 as in FIG. 1 but in a different view, according to an embodiment of the invention. The medical system comprises the internal unit 106 implanted into a body of a patient; a transformer core 112 arranged under the skin 107 of the patient; and internal cabling 108 connecting the internal unit and the transformer core 112, the internal cabling comprising an internal winding around the transformer core. The external unit comprises external cabling 104 including an external winding around the transformer core to allow supply of power from the external unit 109 to the internal unit 106 via the transformer core 112. The external cabling 104 has an openable and closable connector 119. To mount the external unit 109 the external cabling connector is opened and is fed through a pierced healed skin tunnel 110 of a patient 111. Then the connector is closed forming the external winding. After the connector 119 is closed the external unit 109 evaluates a signal indicative of a magnetic flux in the transformer core. Because no other external units are connected the time varying magnetic flux in the transformer core 112 is zero i.e. it is below a predefined threshold flux. Because it is below the threshold flux the external units starts to provide power to the internal unit 106 by the external unit 109 via the transformer core 112.

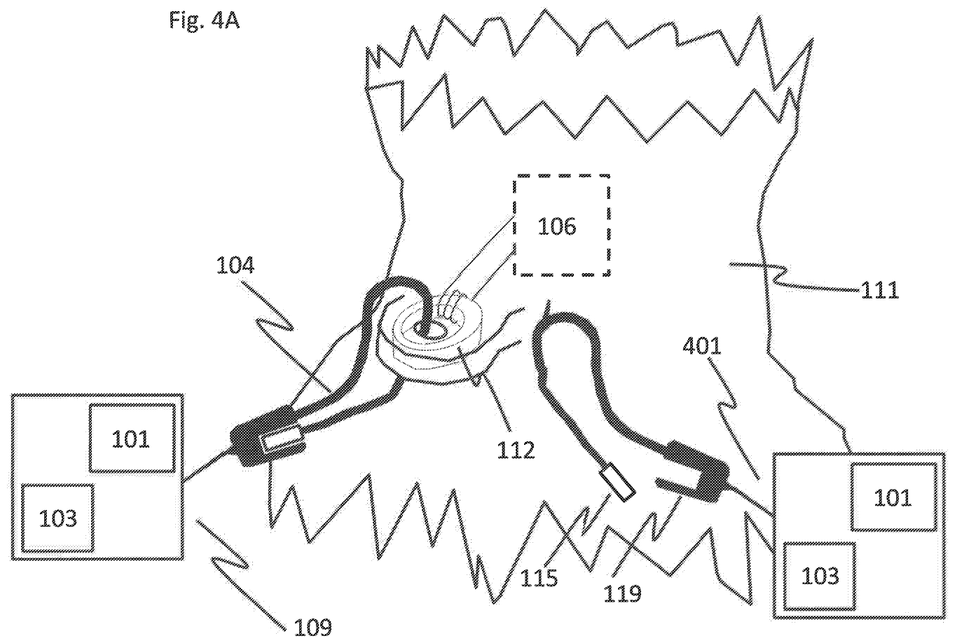

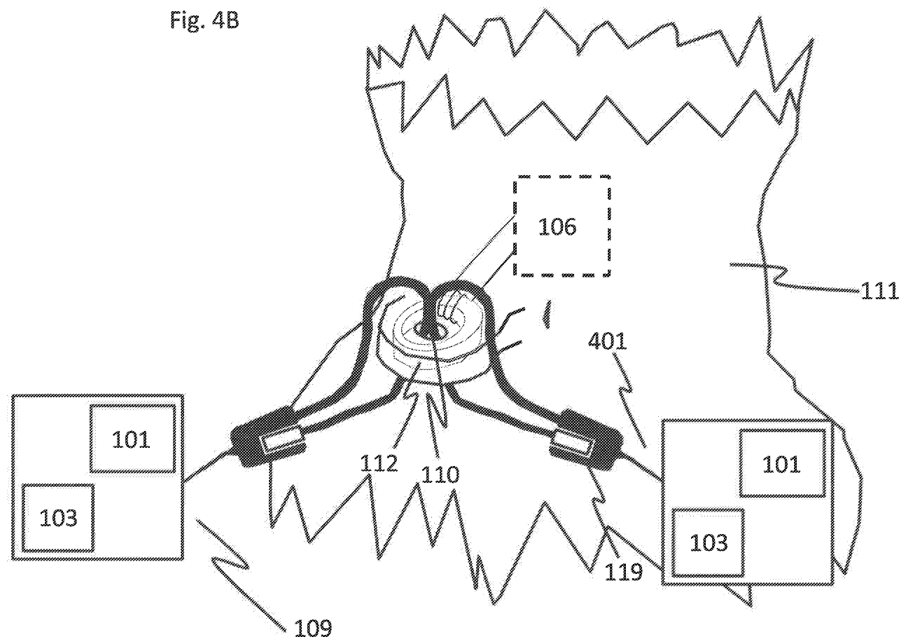

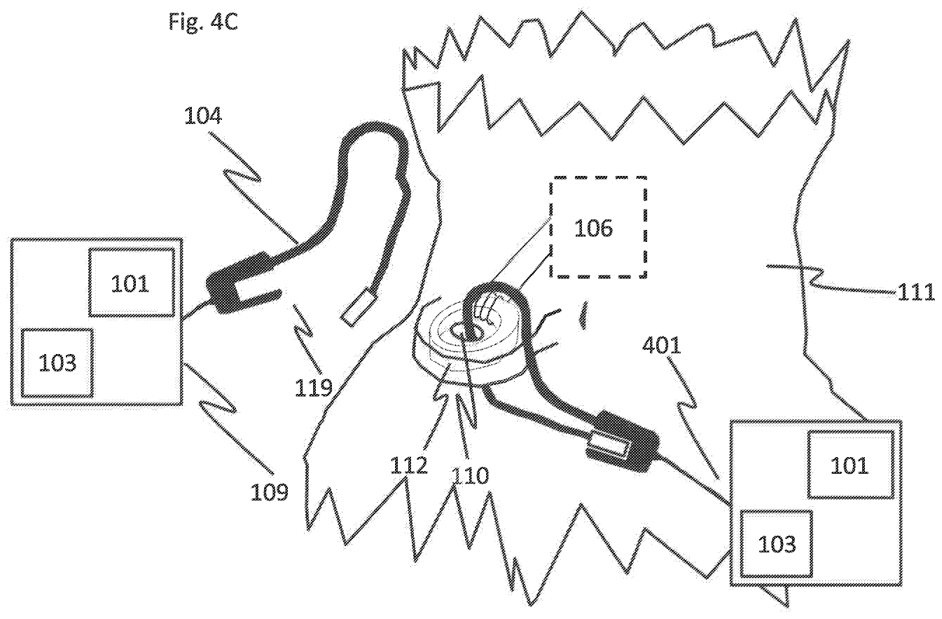

FIG. 4A It will now be explained how a seamless exchange of the external unit is made, according to an embodiment of the invention. A first medical device has an external winding 104, connected to the patient, around the implanted transformer core 112 supplying power from the external unit 109 to the internal unit 106 via the transformer core 112. The power supplied by the external unit 109 is characterized by a time varying current inducing magnetic flux in the transformer core. A secondary external unit 401 with an opened connector 119 is positioned closed to the first external unit.

FIG. 4B The next step is that the second external unit 401 is connected to the patient by feeding the external cabling through the pierced healed skin tunnel 110 of a patient 111. Then the connector 119 is closed forming a secondary external winding around the transformer core. The external unit 109 is at this moment transferring energy from the external unit 109 to the internal unit 106 via the transformer core 112. The secondary external unit 401 is activated and starts to measure and to evaluate a signal indicative of a magnetic flux in the transformer core using the sensing circuitry 101. Since the external unit 109 is transferring energy creating a magnetic flux in the transformer core 112 the secondary unit 401 senses that signal indicating that the magnetic flux in the transformer core is above a predefined threshold flux. Thus, the secondary unit 401 does not provide any power to the internal unit 106 instead it continues to evaluate the signal indicative of a magnetic flux in the transformer core.

FIG. 4C The next step is that the external unit 109 is disconnected by opening the connector 119 and removing the external cabling from the pierced healed skin tunnel 110 of the patient 111. Since the external unit 109 then stops transferring energy there is no more a magnetic flux in the transformer core 112. The secondary unit 401 which measures the signal indicative of a magnetic flux in the transformer core drops below the predefined threshold flux starts to provide power to the internal unit 106 via the transformer core 112. Thus, a seamless switch of external units has been made without any interruption of the power supply to the internal unit.

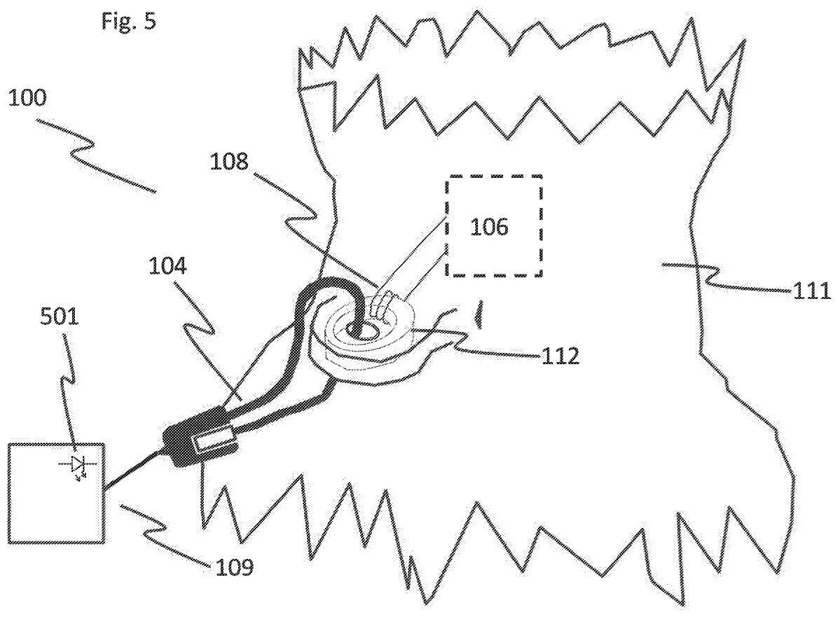

FIG. 5 illustrates the indicator of external unit 109 of a medical system according to an embodiment of the invention. The external unit 109 comprises a LED indicator 501 for providing an indication to an operator or the patient when power is being provided by the external unit to the internal unit.

In the claims, the word “comprising” does not exclude other elements or steps, and the indefinite article “a” or “an” does not exclude a plurality. The mere fact that certain measures are recited in mutually different dependent claims does not indicate that a combination of these measured cannot be used to advantage.

1. A method of initiating operation of an external unit for a medical system further comprising an internal unit implanted into a body of a patient; a transformer core arranged under the skin of the patient; and internal cabling connecting the internal unit and the transformer core, the internal cabling comprising an internal winding around the transformer core, wherein the external unit comprises external cabling including an external winding around the transformer core to allow supply of power from the external unit to the internal unit via the transformer core, the method comprising the steps of:
evaluating, by the external unit, a signal indicative of a magnetic flux in the transformer core;
when the signal indicates that the magnetic flux in the transformer core is below a predefined threshold flux, providing power to the internal unit by the external unit via the transformer core.

2. The method according to claim 1, wherein the step of evaluating is carried out automatically following activation of the external unit.

3. The method according to claim 1, wherein the external unit further comprises sensing circuitry coupled to the external cabling, and the method further comprises the steps of:
activating the sensing circuitry; and
providing, by the sensing circuitry, the signal indicative of the magnetic flux in the transformer core.

4. The method according to claim 1, further comprising the step of:
providing a predefined indication when power is being provided by the external unit to the internal unit.

5. The method according to claim 1, wherein the signal indicative of the magnetic flux in the transformer core is selectively indicative of the magnetic flux within a predefined frequency range.

6. The method according to claim 5, wherein the predefined frequency range has a lower boundary frequency which higher than 5 kHz.

7. The method according to claim 1, comprising the steps of:
activating sensing circuitry comprised in the external unit and configured to sense a property indicative of magnetic flux in the transformer core, and to provide a sensed signal indicative of a sensed value of the property;
acquiring, from the sensing circuitry by processing circuitry comprised in the external unit, the signal indicative of the sensed value;
comparing, by the processing circuitry, the sensed value with a stored value indicative of a threshold magnetic flux; and
controlling, by the processing circuitry, power supply circuitry comprised in the external unit to provide power to the internal unit via the transformer core, when the comparison indicates that the magnetic flux in the transformer core is lower than the threshold magnetic flux.

8. An external unit for a medical system further comprising an internal unit implanted into a body of a patient; a transformer core arranged under the skin of the patient; and internal cabling connecting the internal unit and the transformer core, the internal cabling comprising an internal winding around the transformer core, wherein the external unit comprises:
external cabling configured to allow formation of an external winding around the transformer core;
sensing circuitry coupled to the external cabling and configured to sense a magnetic flux in the transformer core when the external cabling forms the external winding around the transformer core; and
power supply circuitry coupled to the external cabling and configured to supply power to the internal unit via the transformer core when the sensing circuitry is sensing the magnetic flux in the transformer core, and the magnetic flux is below a predefined threshold flux.

9. The external unit according to claim 8, further comprising processing circuitry configured to:
activate the sensing circuitry;
acquire, from the sensing circuitry, a signal indicative of a sensed value indicating the magnetic flux in the transformer core;
compare the sensed value with a stored value indicative of the threshold magnetic flux; and
control the power supply circuitry to supply power to the internal unit via the transformer core when the comparison indicates that the magnetic flux in the transformer core is lower than the threshold magnetic flux.

10. The external unit according to claim 9, wherein:
the external unit further comprises an indicator for providing an indication to an operator; and
the processing circuitry is further configured to control the indicator to provide a predefined indication when power is being provided by the external unit to the internal unit.

11. The external unit according to claim 8, wherein the external cabling comprises:
a connector including a first connector part and a second connector part;
a first conductive current path between the power supply circuitry and the first connector part, conductively connecting the first connector part and the power supply circuitry;
a second conductive current path between the power supply circuitry and the second connector part, conductively connecting the second connector part and the power supply circuitry; and
a third conductive current path between the first connector part and the second connector part, conductively connecting the first connector part and the second connector part,
wherein the first connector part and the second connector part are joinable to conductively connect the first conductive current path to the second conductive current path via the third conductive current path so that a second winding can be formed around the transformer core by joining the first connector part and the second connector part.

12. A medical system comprising:
an internal unit for implantation into a body of a patient;
a transformer core to be arranged under the skin of the patient;
internal cabling connecting the internal unit and the transformer core, the internal cabling comprising an internal winding around the transformer core; and
the external unit according to claim 8.

13. The medical system according to claim 12, wherein the internal unit is a cochlear implant, an auditory transmodiolar implant, an auditory brainstem implant, a bone conduction hearing aid, a middle ear implant, an artificial pacemaker, a blood pumping impeller, a ventricular assist device (VAD), a total artificial heart, an eye implant or retina implant, a nerve stimulator, a deep brain stimulator, a drug delivery system, a brain computer interface system, a cardioverter defibrillator, a gastric stimulator, a brain computer interface system or a rechargeable battery.
